(19) United States
(12) Patent Application Publication
(10) Pub. No.: US 2025/0134887 A1
(43) Pub. Date: May 1, 2025

(54) METHODS OF TREATING PROSTATE CANCER

(71) Applicant: AstraZeneca AB, Södertälje (SE)

(72) Inventor: Jinyu KANG, Gaithersburg, MD (US)

(21) Appl. No.: 18/837,956

(22) PCT Filed: Feb. 14, 2023
(86) PCT No.: PCT/EP2023/053555

§ 371 Date: Aug. 13, 2024

Related U.S. Application Data
(60) Provisional application No. 63/268,026, filed on Feb. 15, 2022.

(51) Int. Cl. A61K 31/502; A61K 31/573; A61K 31/58; A61P 35/04

(52) U.S. Cl. CPC A61K 31/502; A61K 31/573; A61K 31/58; A61P 35/04

(57) ABSTRACT

This disclosure relates to methods of treating prostate cancer in a subject. This disclosure more specifically relates to methods for treating prostate cancer, such as metastatic castration-resistant prostate cancer, by administering to the subject olaparib and abiraterone acetate or a salt thereof.

### BACKGROUND OF THE DISCLOSURE

### Field of the Disclosure

This disclosure relates to methods of treating prostate cancer in a subject. This disclosure more specifically relates to methods for treating prostate cancer, such as metastatic castration-resistant prostate cancer, by administering to the subject olaparib and abiraterone.

### Description of Related Art

Metastatic castration-resistant prostate cancer (mCRPC) is a molecularly heterogeneous disease with poor outcomes. Tumors in up to 30% of patients with mCRPC harbor deleterious DNA damage repair gene aberrations. Among the most common of these alterations, BRCA1 and BRCA2 are well characterized homologous recombination repair (HRR) genes, and ATM functions indirectly to detect DNA damage and activate HRR. Loss-of-function alterations in these and other genes with a direct or indirect role in HRR are associated with more aggressive prostate cancers.

Molecular stratification for treatment is not currently the standard of care for metastatic prostate cancers despite evidence of substantial interpatient genomic heterogeneity. Most therapeutic strategies for advanced prostate cancers target androgen receptor signaling; taxane-based chemotherapies and radiopharmaceuticals are also approved. Although these drugs have improved outcomes in the past decade, metastatic prostate cancer remains invariably fatal and new therapeutic strategies involving molecular stratification are urgently needed.

Genomic studies of metastatic prostate cancer have identified a number of potentially actionable recurrent genomic aberrations, including loss-of-function alterations in DNA repair genes in 20-25% of cases, such as defects in HRR genes. HRR gene alterations confer sensitivity to poly(adenosine diphosphate-ribose) polymerase (PARP) inhibition in prostate and other cancers. Antitumor activity has been reported with the PARP inhibitor, olaparib, in patients with mCRPC harboring HRR gene alterations. Response to PARP inhibition may occur through multiple mechanisms, including PARP trapping, the physical obstruction of replication forks leading to DNA double strand breaks and defects in HRR.

Current treatment options in the first-line setting of mCRPC consist primarily of the next-generation hormonal agents (NHAs) abiraterone and enzalutamide, and taxane-based chemotherapy, depending on the type of treatment that a patient has received following initial diagnosis. Despite the reported clinical activity of these agents, overall survival is approximately 3 years, and the 5-year survival rate is approximately 30%. In the real-world setting approximately 50% of mCRPC patients receive only one life-prolonging therapy.

Therefore, there remains a need for a treatment that provides a significant delay in progression and relapse, and potentially an improvement in cure rates, of prostate cancers.

### SUMMARY OF THE DISCLOSURE

One aspect of the disclosure provides methods for treating prostate cancer in a subject. Such methods include administering to the subject a therapeutically effective amount of 4-[(3-{[4-(cyclopropane-carbonyl)piperazine-1-yl]carbonyl}-4-fluorophenyl)methyl]-2H-phthalazin-1-one (olaparib), or a salt, hydrate, solvate, or prodrug thereof; and administering to the subject a therapeutically effective amount of (3β)-17-(3-pyridinyl) androsta-5,16-dien-3-yl acetate (abiraterone acetate) or a salt, hydrate, or solvate thereof.

Another aspect of the disclosure provides olaparib, or a salt, hydrate, solvate, or prodrug thereof, for use in the treatment of prostate cancer in a subject, wherein said treatment comprises administration of said olaparib, or a salt, hydrate, solvate, or prodrug thereof, and abiraterone acetate or a salt, hydrate, or solvate thereof, to said subject.

In certain embodiments of the methods, uses, and compositions of the disclosure, progression free survival is at least about 6 months greater for the patients receiving olaparib, or a salt, hydrate, solvate, or prodrug thereof, and abiraterone or a salt, hydrate, or solvate thereof, than for subjects receiving abiraterone acetate alone.

In certain embodiments of the methods, uses, and compositions of the disclosure, the prostate cancer is mCRPC and the subject is treatment naïve.

In certain embodiments of the methods, uses, and compositions of the disclosure, the subject has not been selected for homologous recombination repair (HRR) gene mutation(s) in the cancer.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1A provides Kaplan-Meier estimates of imaging-based progression-free survival (PFS) by investigator assessment for the patients in the study provided in the Example.

FIG. 1B provides Kaplan-Meier estimates of imaging-based PFS by blinded independent central review for the patients in the study provided in the Example.

FIG. 2 provides a Forest Plot of prespecified subgroup analysis of imaging-based PFS by investigator assessment for the patients in the study provided in the Example. Analysis performed included the stratification factors selected in the primary pooling strategy as covariates. Each subgroup analysis was performed using a Cox proportional hazards model that contains a term for treatment, factor and treatment by factor interaction. A hazard ratio<1 implies a lower risk of progression in the patient group receiving olaparib and abiraterone acetate. The size of a circle is proportional to the number of events. Subgroup categories with fewer than 5 events in either treatment group have NC presented. *Excludes patients with no baseline assessment. ctDNA, circulating tumor DNA; ECOG, Eastern Cooperative Oncology Group; HRRm, homologous recombination repair gene mutation; mHSPC, metastatic hormone sensitive prostate cancer; NC, non-calculable; PSA, prostate specific antigen.

FIG. 3A provides Kaplan-Meier estimates overall survival by investigator assessment for the patients in the study provided in the Example. In this figure, NR signifies not reached.

FIG. 3B provides Kaplan-Meier estimates time to second progression or death by investigator assessment for the patients in the study provided in the Example.

FIG. 3C provides Kaplan-Meier estimates time to first subsequent therapy or death by investigator assessment for the patients in the study provided in the Example. In this figure, NR signifies not reached.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods, uses, and compositions described herein can be configured by the person of ordinary skill in the art to meet the desired need. The present disclosure provides improvements in treatment of prostate cancer. In certain embodiments, the cancer is prostate cancer that has metastasized. For example, the metastasis is to bone and/or to the lymph nodes. The metastasis may also be visceral. In certain embodiments, the cancer is metastatic castration-resistant prostate cancer (mCRPC). “Metastatic” status is defined as at least one metastatic lesion on either a bone scan, a computed tomography (CT), or magnetic resonance imaging (MRI) scan.

As used herein, the terms “individual,” “patient,” or “subject” are used interchangeably, refers to any animal, including mammals, and most preferably humans.

As provided above, the methods, uses, and compositions of the disclosure as described herein require administration of olaparib. As used herein, “olaparib” refers to the molecule 4-[(3-{[4-(cyclopropane-carbonyl)piperazine-1-yl]carbonyl}-4-fluorophenyl)methyl]-2H-phthalazin-1-one. The molecule olaparib may be employed in the form of a salt, hydrate, solvate, or prodrug thereof. 4-[(3-{[4-(cyclopropane-carbonyl)piperazine-1-yl]carbonyl}-4-fluorophenyl)methyl]-2H-phthalazin-1-one (olaparib), having the following structure, is disclosed in International Publication No. WO 2004/080976 A1, incorporated by reference herein.

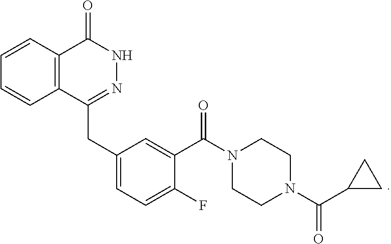 [SMILES: O=C(c1cc(Cc2n[nH]c(=O)c3ccccc23)ccc1F)N1CCN(C(=O)C2CC2)CC1]

Olaparib is administered preferably in the form of a pharmaceutical composition. The therapeutically effective amount of olaparib has been previously established. For example, in certain embodiments, the therapeutically effective amount of olaparib is in the range of about 400 to 800 mg per day. For example, in certain embodiments, olaparib is administered in an amount of about 600 mg daily (e.g., about 300 mg taken twice daily).

As used herein, the phrase “therapeutically effective amount” or “effective amount” refers to the amount of active compound or pharmaceutical agent that elicits the biological or medicinal response that is being sought in a tissue, system, animal, individual or human by a researcher, veterinarian, medical doctor or other clinician.

The methods, uses, and compositions of the disclosure as described herein also require administration of abiraterone, or a salt, hydrate, solvate, or prodrug thereof. An example of a suitable prodrug of abiraterone is abiraterone acetate. As used herein, “abiraterone acetate” refers to the molecule (3β)-17-(3-pyridinyl) androsta-5,16-dien-3-yl acetate (or [(3S,8R,9S,10R,13S,14S)-10,13-dimethyl-17-pyridin-3-yl-2,3,4,7,8,9,11,12,14,15-decahydro-1H-cyclopenta[alphenanthren-3-yl] acetate), having the following structure:

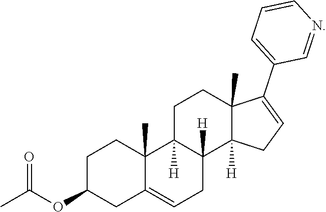

The molecule abiraterone acetate may be employed as a salt, hydrate or solvate.

Abiraterone acetate is administered preferably in the form of a pharmaceutical composition. Therapeutically effective dosages of abiraterone acetate have been previously established. For example, in certain embodiments, the therapeutically effective amount of abiraterone is in the range of about 500 to 1500 mg per day, e.g., about 800 to 1200 mg per day. For example, in certain embodiments, abiraterone acetate is administered in an amount of about 1000 mg daily (e.g., orally once daily). As has been previously established, abiraterone acetate is given in combination with prednisone (5 mg orally twice daily) or prednisolone (5 mg orally twice daily).

The administration of olaparib may be separate, sequential or simultaneous from the administration of abiraterone acetate. In certain embodiments, the administration is simultaneous and/or sequential.

The inventors also unexpectedly found that administering olaparib in combination with abiraterone acetate is sufficient to improve progression free survival (such as imaging-based or radiological progression-free survival, “PFS” or “rPFS”, assessed by Response Evaluation Criteria in Solid Tumors [RECIST 1.1] for soft tissue lesions and/or Prostate Cancer Working Group-3 [PCWG-3] criteria for bone lesions and/or death), or other key therapeutic metric such as overall survival (OS), first subsequent therapy or death (TFST), time to second progression or death (PFS2), objective response rate (ORR), prostate-specific antigen (PSA) response rate and time to PSA progression, in the subject as compared to the subject receiving abiraterone acetate alone (i.e., receiving abiraterone acetate without also receiving olaparib). For example, in the overall subject population, the inventors found that the progression free survival is at least about 6 months greater for subjects receiving olaparib in combination with abiraterone acetate than for subjects receiving abiraterone acetate alone. In certain embodiments, the progression free survival is about 6 to 18 months greater, or about 6 to 14 months greater, or about 6 to 12 months greater. In certain embodiments, the progression free survival is about 8 to 18 months greater, or about 8 to 14 months greater, or about 8 to 12 months greater.

Surprisingly, the improvement in progression free survival was observed for patients regardless of their HRR gene mutation status (i.e., both HRRm and non-HRRm subgroups). Therefore, in certain embodiments, the patient is unselected by HRRm. For example, in certain embodiments, the methods of the disclosure further comprise selecting the subject based on prior treatment and/or selecting the patient does not comprise the step of diagnosing the patient as having cancer cells comprising one or more HRR gene mutations. For example, in certain embodiments, the disclosed methods further comprise selecting the subject without taking into account the HRRm status of the subject's cancer cells.

In certain other embodiments of the methods, uses, and compositions of the disclosure, the prostate cancer is mCRPC that comprises no HRR gene mutations (e.g., no deleterious or suspected deleterious, germline or somatic mutations HRR gene mutation detected in any sample type by tissue analysis, germline, or plasma test result) (also as “non-HRRm” herein).

Another embodiment of the disclosure provides methods, uses, and compositions where the prostate cancer is homologous recombination deficient (HRD) cancer. For example, whether the cancer is HRD positive can be determined by Myriad Genetics myChoice® HRD, myChoice® HRD Plus, or a suitable equivalent assay. In certain other embodiments, the methods of the disclosure further comprise identifying the subject as having cancer cells comprising one or more HRR gene mutations.

In certain other embodiments of the methods, uses, and compositions of the disclosure, the prostate cancer comprises one or more HRR gene mutations (also as “HRRm” herein). As used herein, the HRR gene mutation includes deleterious or suspected deleterious, germline or somatic mutations, detected in one or more of the samples analysed, including tissue, germline, and plasma.

In certain embodiments, the cancer cells comprise HRR gene mutation selected from BRCA1, BRCA2, ATM, BRIP1, BARD1, CDK12, CHEK1, CHEK2, FANCL, PALB2, PPP2R2A, RAD51B, RAD51C, RAD51D, and RAD54L gene mutation. In certain embodiments, the cancer cells comprise a BRCA1, a BRCA2, and/or an ATM gene mutation. In certain other embodiments, the methods, uses, and compositions of the disclosure further comprise identifying the subject having cancer cells comprising a BRCA1, a BRCA2, and/or an ATM gene mutation. In certain embodiments, the cancer cells comprise a BRCA1 and/or a BRCA2 gene mutation. In certain embodiments, the cancer cells comprise an ATM gene mutation. In certain embodiments, the cancer cells comprise a BRIP1, a BARD1, a CDK12, a CHEK1, a CHEK2, a FANCL, a PALB2, a PPP2R2A, a RAD51B, a RAD51C, a RAD51D, and/or a RAD54L gene mutation.

In certain other embodiments of the methods, uses, and compositions of the disclosure, the prostate cancer is mCRPC comprising one or more HRR gene mutations.

In certain embodiments, wherein the subject is treatment naïve, as defined below, the progression free survival is about 6 to 18 months greater (e.g., about 6 to 14 months greater, or about 6 to 12 months greater, or about 8 to 18 months greater, or at least about 6 months greater, or at least about 8 months greater).

Thus, in certain embodiments, the methods, uses, and compositions of the disclosure are also useful as a first line treatment, wherein the subject is treatment naïve. “Treatment naïve” subject, as used herein, is the subject that has not previously received or completed any cytotoxic chemotherapy (such as a first line platinum- and/or taxane-based chemotherapy or docetaxel), and/or new hormonal agent (NHA) chemotherapy (such as enzalutamide or abiraterone) or any other systemic treatment (approved drugs or experimental compounds) for prostate cancer, such as for mCRPC. In certain embodiments, the treatment naïve subject previously received docetaxel during localized prostate cancer treatment stage or for metastatic hormone-sensitive prostate cancer (mHSPC) stage. In certain embodiments, treatment naïve subject has not previously received abiraterone, or a salt, hydrate, solvate or ester thereof (such as abiraterone acetate) for prostate cancer, e.g., during any treatment stage.

In certain other embodiments of the methods, uses, and compositions of the disclosure, the prostate cancer is mCRPC, and the subject has not previously received docetaxel.

In certain embodiments, wherein the subject received prior chemotherapy (such as docetaxel) during neoadjuvant/adjuvant treatment for localized prostate cancer and/or the metastatic hormone-sensitive (mHSPC) stage of disease, the progression free survival is about 8 to 24 months greater (e.g., about 8 to 20 months greater, or about 8 to 18 months greater, or at least about 8 months greater, or at least about 10 months greater, or at least about 12 months greater).

The methods, uses, and compositions of disclosure are thus also useful as a second line treatment, wherein the subject has previously received a first line of therapy. The methods, uses, and compositions of the disclosure, in certain embodiments, may provide a delay in progression and relapse of cancer of subjects that have previously received or completed a first line of chemotherapy. For example, in certain embodiments, the subject has previously received or completed a first line platinum- and/or taxane-based chemotherapy, such as docetaxel. In certain other embodiments, the subject previously received or completed NHA chemotherapy, such as enzalutamide or abiraterone.

In certain other embodiments of the methods, uses, and compositions of the disclosure, the prostate cancer is mCRPC, and the subject has previously received docetaxel.

### Example

The methods, uses, and compositions of the disclosure are illustrated further by the following example, which is not to be construed as limiting the disclosure in scope or spirit to the specific procedures and compounds described in them.

### Study Design and Objective

The efficacy of olaparib vs. enzalutamide or abiraterone was evaluated in PROfound (NCT02987543), randomized, open-label, multi-center trial. The PROfound trial demonstrated imaging-based progression-free survival and overall survival benefits with olaparib in patients with mCRPC harboring BRCA1, BRCA2 and ATM mutations whose disease had progressed on NHA. Imaging-based progression-free survival and a trend towards prolonged overall survival in patients in the overall trial population with alterations in HRR genes were also observed.

The rationale for the present study is based on preclinical models that suggest that when PARP inhibitors are combined with NHAs there may be a combined anti-tumor effect for patients regardless of whether they have an HRR gene mutation (HRRm). Without being bound by a theory, it is believed that this is potentially due to PARP involvement in positive co-regulation of androgen receptor (AR) signaling, which leads to enhanced AR target gene suppression when PARP/AR signaling is co-inhibited. Some studies have reported that NHAs inhibit the transcription of some HRR genes; therefore, inducing HRR deficiency and increased sensitivity to PARP inhibitors via non-genetic mechanisms.

The design of the present study was put into practice as the PROpel trial (NCT03732820), which was a double-blind randomized phase Ill trial of abiraterone acetate and olaparib in the first-line treatment of patients with mCRPC, and described in this example. Eligible patients were randomized to receive abiraterone acetate and olaparib or abiraterone acetate and placebo. The primary objective was efficacy as assessed by investigator-assessed imaging-based progression-free survival. The claimed embodiments are based on data and observations arising during the PROpel trial.

### Methods

Patient population. Eligible patients were 18 years of age (or 19 years of age in South Korea) and had histologically or cytologically confirmed prostate adenocarcinoma with at least one documented metastatic lesion on either a bone scan, computed tomography, or magnetic resonance imaging scan. With the exception of androgen depletion therapy (ADT), and first-generation anti-androgen agents (e.g., bicalutamide, nilutamide, and flutamide) with a 4-week washout period, prior systemic treatment in the mCRPC first-line setting was not allowed (i.e., patients were treatment naïve at mCRPC stage, e.g., patients should not have received any cytotoxic chemotherapy, NHA, or other systemic treatment (approved drugs or experimental compounds) in the mCRPC setting). Docetaxel during neoadjuvant/adjuvant treatment for localized prostate cancer and the metastatic hormone-sensitive (mHSPC) stage of disease was permitted, as long as no signs of failure or disease progression occurred during or immediately after such treatment. Prior to mCRPC stage, treatment with second-generation antiandrogen agents (except abiraterone) without PSA progression/clinical progression/radiographic progression during treatment was allowed, provided the treatment was stopped at least 12 months before randomisation.

Trial design and interventions. This was a double-blind, placebo-controlled, phase Ill trial. Eligible patients were randomized (1:1) to treatment with abiraterone acetate (1000 mg once daily) in combination with either olaparib (300 mg twice daily) or placebo. All patients received prednisone or prednisolone (5 mg twice daily) as per the abiraterone acetate label requirement. Randomization was stratified by distant metastasis type (bone only/visceral/other) at baseline and by docetaxel treatment at the mHSPC stage of disease (yes or no). Study treatment continued until objective imaging-based progressive disease assessed by investigator (using Response Evaluation Criteria in Solid Tumors [RECIST 1.1] for soft tissue lesions and Prostate Cancer Working Group-3 [PCWG-3] criteria for bone lesions), unacceptable toxicity, or withdrawal of consent. Following objective disease progression, further treatment options were at the discretion of the investigator. Patients could continue study treatment if the investigator believed, that the patient could continue to receive clinical benefit, was not experiencing serious toxicity and there was no available better alternative treatment. Crossover from placebo to receive olaparib in combination with abiraterone acetate was not allowed.

Endpoints. The primary endpoint was imaging-based progression-free survival or death from any cause in the absence of disease progression. Sensitivity analysis by blinded independent central review and exploratory subgroup analysis of investigator-assessed imaging-based progression-free survival to assess consistency of treatment effect across potential prognostic factors were prespecified. Subgroups included HRRm status.

A key secondary endpoint was overall survival. Other secondary endpoints included time to first subsequent therapy or death (TFST), time to second progression or death (PFS2) and patient-reported outcomes. Further exploratory endpoints were objective response rate (ORR), prostate-specific antigen (PSA) response rate and time to PSA progression.

Safety was assessed through reporting of adverse events and serious adverse events (according to Common Terminology Criteria for Adverse Events v.4.03) based on physical examination findings, vital signs, ECG findings and laboratory test results.

Statistical analysis. Efficacy was analyzed for the intention-to-treat population and safety was analyzed for all patients who received any amount of abiraterone acetate, olaparib, or placebo. Patients who received at least one dose of olaparib were included in the abiraterone acetate and olaparib arm.

The first planned interim analysis of the primary endpoint of imaging-based progression-free survival is reported in this example. Imaging-based progression-free survival will be reported subsequently at a second data cutoff. Overall survival will be formally tested at all points, including a third data cutoff.

With a sample size of 796 patients, the first interim analysis was planned to occur when there had been approximately 379 progression or death events (47.6% maturity), to provide 94.1% power at a one-sided alpha of 0.014 to show a statistically significant difference in imaging-based progression-free survival between the trial arms, assuming a hazard ratio for progression or death of 0.68.

A multiple testing procedure controlled the overall one-sided type 1 error rate of 2.5%. If the primary endpoint of imaging-based progression-free survival was statistically significant, then overall survival would be tested in a hierarchical fashion.

For time-to-event endpoints, a stratified log-rank test was used to calculate two-sided P values. Hazard ratios and 95% confidence intervals (CIs) were calculated using the Cox proportional hazards model including the two stratification variables as covariates. Kaplan-Meier plots were used to calculate medians.

### Results

Screening and randomization. This multicenter trial spanning 17 countries screened 1103 patients; 796 patients met eligibility criteria and were randomized.

Patient characteristics. In total, 399 patients were assigned to abiraterone acetate and olaparib and 397 to abiraterone acetate and placebo. Baseline characteristics were generally well balanced between the arms (Table 1). Based on ctDNA testing 67.3% of patients were included in the non-HRRm subgroup.

TABLE 1
Characteristics of Patients at Baseline

| | Abiraterone and | | Abiraterone and | |
|---|---|---|---|---|
| | Olaparib (n = 399) | | Placebo (n = 397) | |
| Median age at randomization, | 69.0 | (43-91) | 70.0 | (46-88) |
| years (range) | | | | |
| Age >65 years at randomization, n (%) | 269 | (67.4) | 300 | (75.6) |
| Race, n (%) | | | | |
| White | 282 | (70.7) | 275 | (69.3) |
| Asian | 66 | (16.5) | 72 | (18.1) |
| Black or African American | 14 | (3.5) | 11 | (2.8) |
| Other | 15 | (3.8) | 9 | (2.3) |
| Missing | 22 | (5.5) | 30 | (7.6) |
| Histology, adenocarcinoma, n (%) | 398 | (99.7) | 397 | (100) |
| Gleason score, n (%) | | | | |
| ≥8 | 265 | (66.4) | 258 | (65.0) |
| Missing | 13 | (3.3) | 5 | (1.3) |
| Distant metastasis at initial | 257 | (64.4) | 242 | (61.0) |
| diagnosis, n (%) | | | | |
| Missing | 1 | (0.3) | 1 | (0.3) |
| ECOG, n (%) | | | | |
| (0) Normal activity | 286 | (71.7) | 272 | (68.5) |
| (1) Restricted activity | 112 | (28.1) | 124 | (31.2) |
| Missing | 1 | (0.3) | 1 | (0.3) |
| Type of progression, n (%) | | | | |
| PSA progression | 172 | (43.1) | 173 | (43.6) |
| Imaging-based progression | 92 | (23.1) | 73 | (18.4) |
| Both | 134 | (33.6) | 150 | (37.8) |
| Missing | 1 | (0.3) | 1 | (0.3) |
| Prior docetaxel treatment | | | | |
| Yes | 97 | (24.3) | 98 | (24.7) |
| At mHSPC stage | 90 | (22.6) | 89 | (22.4) |
| Prior treatment with NHA*, n (%) | | | | |
| Yes | 1 | (0.3) | 0 | |
| Prior local treatment with curative | | | | |
| intent, n (%) | | | | |
| Yes | 134 | (33.6) | 144 | (36.3) |
| Baseline pain score (BPI-SF Item 3 | | | | |
| score)^{†}, n (%) | | | | |
| 0 (no pain) | 133 | (33.3) | 137 | (34.5) |
| >0-<4 (mild pain) | 151 | (37.8) | 173 | (43.6) |
| 4-<6 (moderate pain) | 53 | (13.3) | 36 | (9.1) |
| 6-10 (severe pain) | 32 | (8.0) | 28 | (7.1) |
| Missing | 30 | (7.5) | 23 | (5.8) |
| Site of disease^{‡}, n (%) | | | | |
| Bone | 349 | (87.5) | 339 | (85.4) |
| Distant lymph nodes | 133 | (33.3) | 119 | (30.0) |
| Locoregional lymph nodes | 82 | (20.6) | 89 | (22.4) |
| Prostate and adjacent structures | 47 | (11.8) | 46 | (11.6) |
| Respiratory (including lung) | 40 | (10.0) | 42 | (10.6) |
| Liver | 15 | (3.8) | 18 | (4.5) |
| HRRm status^{§} | | | | |
| Based on ctDNA, n (%) | | | | |
| HRRm | 98 | (24.6) | 100 | (25.2) |
| Non-HRRm | 269 | (67.4) | 267 | (67.3) |
| HRRm unknown | 32 | (8.0) | 30 | (7.6) |
| Based on tissue test, n (%) | | | | |
| HRRm | 62 | (15.5) | 56 | (14.1) |
| Non-HRRm | 207 | (51.9) | 210 | (52.9) |
| HRRm unknown | 130 | (32.6) | 131 | (33.0) |
| Median time from initial diagnosis to | 33.6 | (4-288) | 39.5 | (1-279) |
| randomization, months (range) | | | | |
| Baseline S-prostate specific antigen, | 17.90 | (6.09-67.00) | 16.81 | (6.26-53.30) |
| median (ug/L), (IQR) | | | | |
| S/P-alkaline phosphatase, median | 1.88 | (1.35-3.38) | 1.83 | (1.30-3.35) |
| ukat/L (IQR) | | | | |
| Baseline S/P-lactate dehydrogenase, | 3.63 | (3.07-4.69) | 3.50 | (2.93-4.42) |
| median ukat/L (IQR) | | | | |
| Baseline hemoglobin, median g/L, | 132.0 | (123.0-139.0) | 133.0 | (124.0-140.0) |
| (IQR) | | | | |

*Treatment with abiraterone in the hormone-naïve state was not allowed, although treatment with other NHAs was permitted, as long as patients had not had prostate-specific antigen, clinical, or radiological progression during that treatment, and provided the treatment was stopped at least 12 months before randomization.
^{†}Baseline pain score is based on a patient completing the BPI-SF questionnaire item three (worst pain) at least once during the seven day baseline period and is presented as an average.
^{‡}Investigators could select more than one site of disease. Entries for “Other locally advanced sites”, “Other distant sites” and “Other” have been excluded.
^{§}HRRm: Any deleterious or suspected deleterious HRR gene mutation detected; Non-HRRm: No deleterious or suspected deleterious HRR gene mutation detected; HRRm unknown: Patients for whom mutation testing was not performed or where mutation testing failed due to insufficient quantity or quality of sample, or technical failure at sequencing or post-sequencing steps on analysis

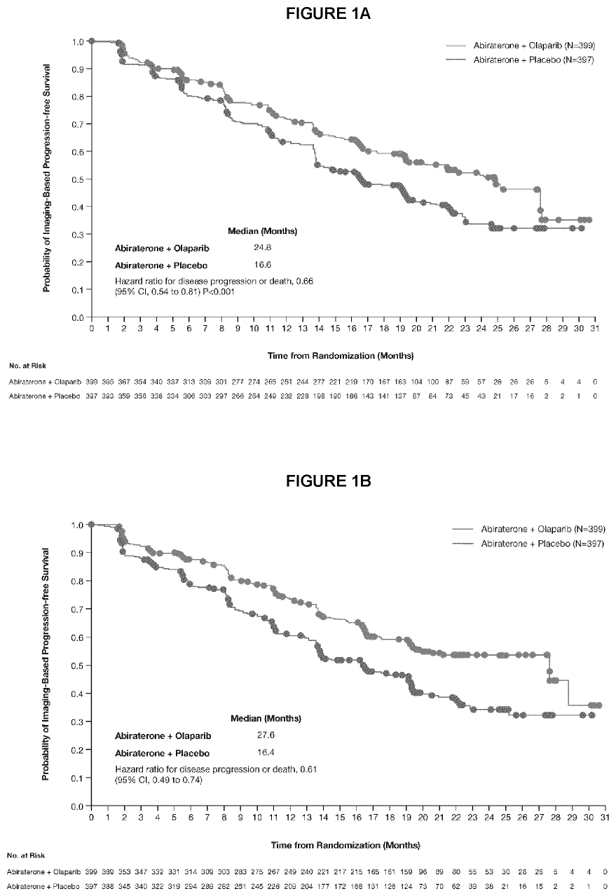

Efficacy. Analysis occurred after 394 patients had an imaging-based progression event or had died (49.5% maturity, data cutoff Jul. 30, 2021). The median imaging-based progression-free survival by investigator assessment was significantly longer for the abiraterone acetate and olaparib arm than for the abiraterone acetate and placebo arm (24.8 vs. 16.6 months; hazard ratio [HR]0.66; 95% CI, 0.54 to 0.81; P<0.001) (FIG. 1A). There were 168 progression or death events in abiraterone acetate and olaparib arm and 226 in the abiraterone acetate and placebo arm. The median (range) duration of follow-up for disease progression in patients with censored data was 19.3 (0.03-30.59) months in the abiraterone acetate and olaparib arm and 19.4 (0.03-30.16) months in the abiraterone acetate and placebo arm.

Prespecified sensitivity analysis of imaging-based progression-free survival by blinded independent central review was consistent with the results of the primary analysis (median 27.6 vs. 16.4 months; HR, 0.61; 95% CI, 0.49 to 0.74) (FIG. 1B). There were 157 progression or death events in abiraterone acetate and olaparib arm and 218 in the abiraterone acetate and placebo arm. The median (range) duration of follow-up for disease progression in patients with censored data was 19.3 (0.03-30.59) months in the abiraterone acetate and olaparib arm and 19.2 (0.03-30.16) months in the abiraterone acetate and placebo arm.

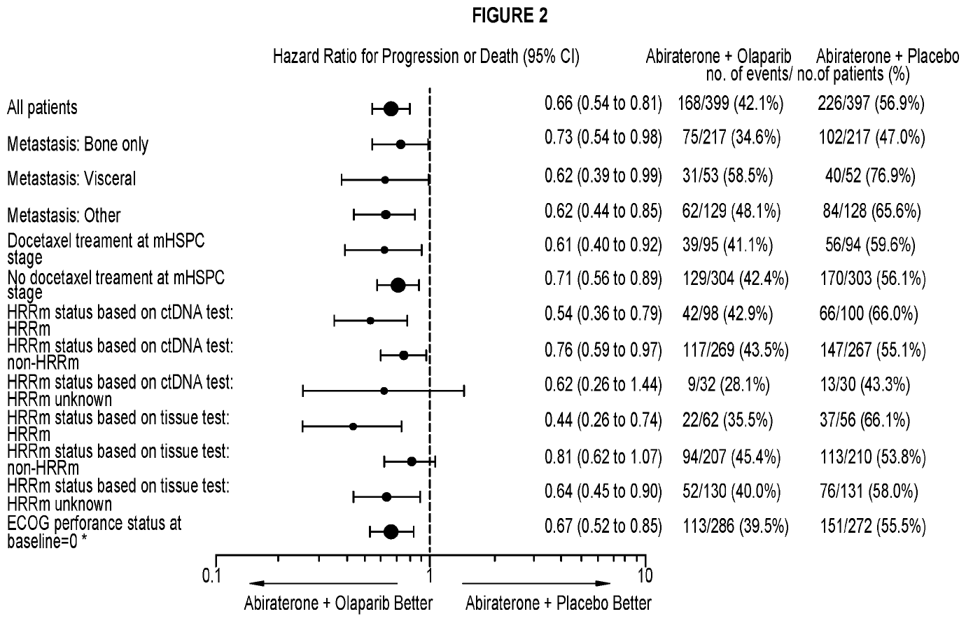

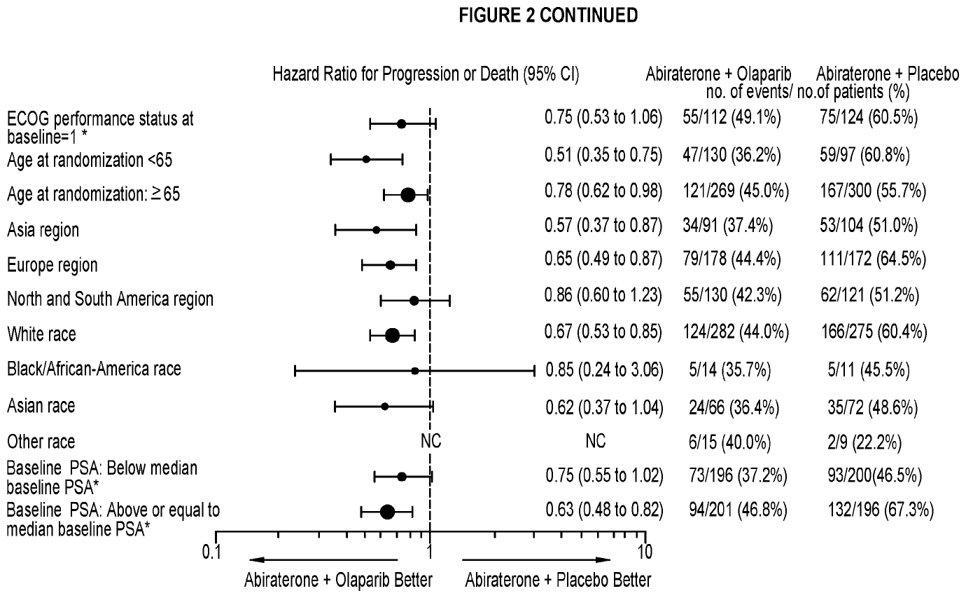

An imaging-based progression-free survival benefit was observed across all prespecified subgroups (FIG. 2). A global interaction test comparing the fit of a model with no interaction terms to a model with all subgroup interactions included, was not significant at the 10% level (P=0.41), indicating a consistent treatment effect between subgroups.

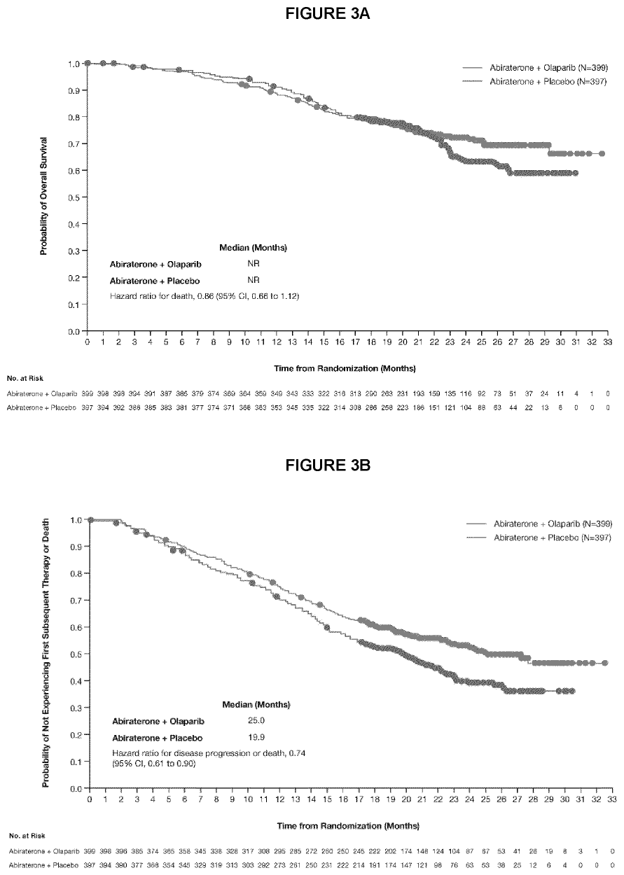

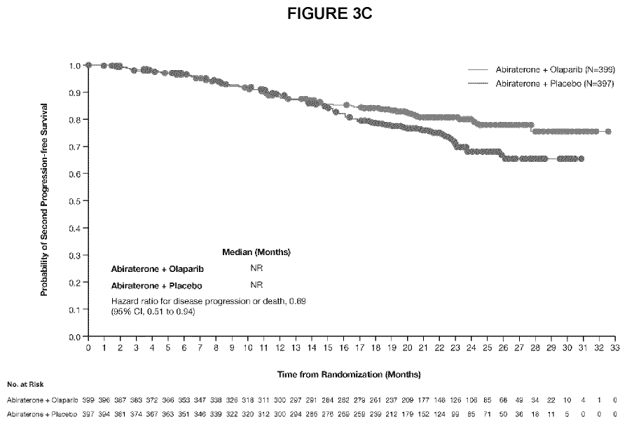

Although the maturity of the overall survival data was 28.6% there was a trend towards prolonged overall survival (HR 0.86; 95% CI, 0.66 to 1.12) (FIG. 3A). There were 107 deaths in the abiraterone acetate and olaparib arm and 121 in the abiraterone acetate and placebo arm. The median (range) duration of follow-up for disease progression in patients with censored data was 22.2 (0.03-32.56) months in the abiraterone acetate and olaparib arm and 21.8 (0.10-30.88) months in the abiraterone acetate and placebo arm.

TFST (HR 0.74; 95% CI, 0.61 to 0.90) and PFS2 (HR 0.69; 95% CI, 0.51 to 0.94) were supportive of an efficacy benefit beyond first imaging-based progression (FIGS. 3B and 3C).

For TFST, there were 183 patients who received a first subsequent therapy in the abiraterone acetate and olaparib arm and 221 patients in the abiraterone acetate and placebo arm. Time to first subsequent therapy (excluding radiotherapy) was defined as the time from randomization to the start date of the first subsequent anti-cancer therapy after discontinuation of randomized treatment or death from any cause (whichever was earlier). Any patient not known to have died at the time of the analysis and not known to have had a subsequent therapy was censored at the last known time to have not received first subsequent therapy.

For PFS2, there were 70 second progression or death events in the abiraterone acetate and olaparib arm and 94 in the abiraterone acetate and placebo arm. Patients who had not had a second disease progression event or died at the time of analysis, or who had second progression or died after two or more missed visits, were censored at the latest evaluable assessment when they were known to be alive and without a second disease progression.

Of patients with measurable disease at baseline, the ORR was 58.4% (94 of 161 patients) in the abiraterone acetate and olaparib arm vs. 48.1% (77 of 160 patients) in the abiraterone acetate and placebo arm (odds ratio, 1.60; 95% CI, 1.02 to 2.53).

Confirmed PSA response was 79.3% in the abiraterone acetate and olaparib arm and 69.2% in the abiraterone acetate and placebo arm. Median time to PSA progression was not reached versus 12.0 months in the abiraterone acetate and olaparib and the abiraterone acetate and placebo arms, respectively (HR 0.55, 95% CI 0.45 to 0.68).

Patient-reported outcomes. Least-square mean change from baseline in FACT-P (Functional Assessment of Cancer Therapy-Prostate) Total Score across all visits was −4.85 in the abiraterone acetate and olaparib arm vs. −4.03 in the abiraterone acetate and placebo arm (difference −0.82; 95% CI, −3.56 to 1.92), suggesting that there was no detriment to health-related quality of life (HRQoL) when olaparib was added to abiraterone acetate treatment.

Safety. At the data cutoff, the median total duration of exposure was 17.5 months for olaparib, 15.7 months for placebo, 18.2 months for abiraterone acetate in the abiraterone acetate and olaparib arm and 15.7 months for abiraterone acetate in the abiraterone acetate and placebo arm.

The three most common adverse events in the abiraterone acetate and olaparib arm were anemia, nausea, and fatigue. Anemia was the most common Grade≥3 adverse event, occurring in 60 patients (15.1%) in the abiraterone acetate and olaparib arm and 13 patients (3.3%) in the abiraterone acetate and placebo arm.

The rate of cardiovascular events (myocardial infarction, congestive heart failure, and ischemic stroke) was similar between the treatment arms. See Table 2 for further details.

There were 26 cases (6.5% of patients) of pulmonary embolism in the abiraterone acetate and olaparib arm and seven (1.8% of patients) in the abiraterone acetate and placebo arm; one event in the abiraterone acetate and olaparib arm was fatal but was considered to be unrelated to study treatment (more information in Supplement). Pulmonary embolism led to olaparib dose interruption in eight (2.0%) patients and abiraterone acetate dose interruption in six (1.5%) patients in the abiraterone acetate and olaparib arm; no events led to treatment discontinuation. Deep-vein thrombosis occurred in seven (1.8%) and three (0.8%) patients in the abiraterone acetate and olaparib and abiraterone acetate and placebo, arms respectively.

Development of myelodysplastic syndrome or acute myeloid leukemia was not reported. There were 12 reports (3.0% of patients) of new primary cancers in the abiraterone acetate and olaparib arm and 10 reports (2.5% of patients) in the abiraterone acetate and placebo arm. Three (0.8%) patients in each arm of the trial had pneumonitis (one in each arm had interstitial lung disease).

There were 33 cases (8.3% of patients) of COVID-19 in the abiraterone acetate and olaparib arm and 18 cases (4.5% of patients) in the abiraterone acetate and placebo arm of the trial, the majority of patients were unvaccinated and were from Brazil, Turkey and the USA. Grade≥3 cases occurred in 17 (4.3%) and 8 (2.0%) of abiraterone acetate and olaparib and abiraterone acetate and placebo patients, respectively.

TABLE 2
Treatment-Emergent Adverse Events (Safety Analysis Set).*

| | Abiraterone and Olaparib | | | | Abiraterone and Placebo | | | |
|---|---|---|---|---|---|---|---|---|
| | (n = 398) | | | | (n = 396) | | | |
| Adverse Event | All Grades | | Grade ≥ 3 | | All Grades | | Grade ≥ 3 | |
| Any adverse event | 387 | (97.2) | 188 | (47.2) | 376 | (94.9) | 152 | (38.4) |
| Anemia^{†} | 183 | (46.0) | 60 | (15.1) | 65 | (16.4) | 13 | (3.3) |
| Fatigue or asthenia | 148 | (37.2) | 9 | (2.3) | 112 | (28.3) | 6 | (1.5) |
| Nausea | 112 | (28.1) | 1 | (0.3) | 50 | (12.6) | 1 | (0.3) |
| Diarrhea | 69 | (17.3) | 3 | (0.8) | 37 | (9.3) | 1 | (0.3) |
| Constipation | 69 | (17.3) | 0 | | 55 | (13.9) | 1 | (0.3) |
| Back pain | 68 | (17.1) | 3 | (0.8) | 73 | (18.4) | 4 | (1.0) |
| Decreased appetite | 58 | (14.6) | 4 | (1.0) | 23 | (5.8) | 0 | |
| Vomiting | 52 | (13.1) | 4 | (1.0) | 36 | (9.1) | 1 | (0.3) |
| Arthralgia | 51 | (12.8) | 0 | | 70 | (17.7) | 2 | (0.5) |
| Hypertension | 50 | (12.6) | 14 | (3.5) | 65 | (16.4) | 13 | (3.3) |
| Dizziness | 43 | (10.8) | 0 | | 25 | (6.3) | 0 | |
| Peripheral edema | 41 | (10.3) | 0 | | 45 | (11.4) | 1 | (0.3) |
| Urinary tract | 41 | (10.3) | 8 | (2.0) | 31 | (7.8) | 4 | (1.0) |
| infection | | | | | | | | |
| Cardiac failure adverse | 6 | (1.5) | NA | | 5 | (1.3) | NA | |
| events | | | | | | | | |
| Embolic and thrombotic | 8 | (2.0) | NA | | 10 | (2.5) | NA | |
| arterial adverse events | | | | | | | | |
| Any serious adverse event | 135 | (33.9) | NA | | 107 | (27.0) | NA | |
| Interruption of | 178 | (44.7) | NA | | 100 | (25.3) | NA | |
| olaparib/placebo because | | | | | | | | |
| of an adverse event | | | | | | | | |
| Interruption of abiraterone | 131 | (32.9) | NA | | 87 | (22.0) | NA | |
| acetate because of an | | | | | | | | |
| adverse event | | | | | | | | |
| Dose reduction of | 80 | (20.1) | NA | | 22 | (5.6) | NA | |
| olaparib/placebo due | | | | | | | | |
| to an adverse event | | | | | | | | |
| Dose reduction of | 10 | (2.5) | NA | | 17 | (4.3) | NA | |
| abiraterone acetate | | | | | | | | |
| due to an adverse event | | | | | | | | |
| Discontinuation of | 55 | (13.8) | NA | | 31 | (7.8) | NA | |
| olaparib/placebo due | | | | | | | | |
| to an adverse event | | | | | | | | |
| Discontinuation of | 34 | (8.5) | NA | | 35 | (8.8) | NA | |
| abiraterone acetate | | | | | | | | |
| due to an adverse event | | | | | | | | |
| Death due to an adverse | 16 | (4.0) | NA | | 17 | (4.3) | NA | |
| event | | | | | | | | |

*Adverse events, regardless of the investigators' assessment of causality, are reported for those that occurred in at least 10% of the patients in either treatment group. Patients were counted once for each type of adverse event. Adverse events with an onset date, or worsening, on or after the date of first dose and up to and including 30 days following discontinuation of randomized treatment are included.
^{†}Anemia category includes anemia, decreased hemoglobin level, decreased red-cell count, decreased hematocrit level, erythropenia, macrocytic anemia, normochromic anemia, normochromic normocytic anemia, and normocytic anemia.
NA, not applicable

### Discussion

At interim analysis, the present study met its primary objective of increased imaging-based progression-free survival when abiraterone and olaparib was used by comparison with abiraterone acetate and placebo in patients receiving first line-treatment for mCRPC. The delay in imaging-based progression-free survival was clinically relevant (˜8 to 11 months longer than for abiraterone acetate and placebo), is the longest reported to date in this population, and exceeds the median overall survival reported in phase Ill docetaxel trials.

The active control arm of abiraterone acetate plus prednisone/prednisolone and placebo performed as expected; in the COU-AA-302 study in patients with progressive mCRPC who had not received chemotherapy, median radiographic progression-free survival with abiraterone acetate was 16.5 months (Ryan C J et al. Lancet Oncol 2015; 16:152-60). This compares with the 16.6 months reported herein, illustrating that combining abiraterone acetate and olaparib treatment significantly extended imaging-based progression-free survival beyond the present standard of care.

There was a clinically meaningful improvement in imaging-based progression-free survival in all prespecified subgroups, including subgroups for prior and no prior treatment with docetaxel at mHSPC stage of disease, type of metastasis at baseline and HRRm status. Summary of results is provided in Table 3.

TABLE 3
Radiological PFS (investigator assessment) cox proportional
hazards exploratory subgroup analyses (FAS)

| | Olaparib 300 mg bd + | Placebo bd + |
|---|---|---|
| | abiraterone acetate | abiraterone |
| Subgroup | 1000 mg qd | acetate 1000 mg qd |

All patients^{a}

| | | | | | |
|---|---|---|---|---|---|
| All patients | Number of events^{b}/total | 168/399 | (42.1) | 226/397 | (56.9) |
| | number of patients (%) | | | | |
| | Median rPFS (months) (95% CI) | 24.84 | (20.47, 27.63) | 16.59 | (13.93, 19.22) |

| | |
|---|---|
| HR (95% CI) | 0.66 (0.54, 0.81) |

Stratification factors at randomisation (IxRS)

| Site of distant | Number of events^{b}/total | 75/217 | (34.6) | 102/217 | (47.0) |
|---|---|---|---|---|---|
| metastases: | number of patients (%) | | | | |
| Bone only | Median rPFS (months) (95% CI) | 27.63 | (24.11, NC) | 22.18 | (19.12, NC) |
| | HR (95% CI) | 0.73 (0.54, 0.98) | | | |
| Site of distant | Number of events^{b}/total | 31/53 | (58.5) | 40/52 | (76.9) |
| metastases: | number of patients (%) | | | | |
| Visceral | Median rPFS (months) (95% CI) | 13.73 | (8.57, NC) | 10.91 | (5.29, 13.80) |
| | HR (95% CI) | 0.62 (0.39, 0.99) | | | |
| Site of distant | Number of events^{b}/total | 62/129 | (48.1) | 84/128 | (65.6) |
| metastases: | number of patients (%) | | | | |
| Other | Median rPFS (months) (95% CI) | 20.47 | (16.59, 27.66) | 13.70 | (11.07, 16.36) |
| | HR (95% CI) | 0.62 (0.44, 0.85) | | | |
| Docetaxel | Number of events^{b}/total | 39/95 | (41.1) | 56/94 | (59.6) |
| treatment at | number of patients (%) | | | | |
| mHSPC stage | Median rPFS (months) (95% CI) | 27.60 | (16.46, NC) | 13.83 | (10.91, 19.19) |
| | HR (95% CI) | 0.61 (0.40, 0.92) | | | |
| No docetaxel | Number of events^{b}/total | 129/304 | (42.4) | 170/303 | (56.1) |
| treatment at | number of patients (%) | | | | |
| mHSPC stage | Median rPFS (months) (95% CI) | 24.84 | (20.47, 27.63) | 16.82 | (14.75, 19.45) |

| | |
|---|---|
| HR (95% CI) | 0.71 (0.56, 0.89) |

Baseline Characteristics

| Age at | Number of events^{b}/total | 47/130 | (36.2) | 59/97 | (60.8) |
|---|---|---|---|---|---|
| randomisation: | number of patients (%) | | | | |
| <65 years | Median rPFS (months) (95% CI) | NC | (NC, NC) | 16.36 | (11.70, 20.27) |
| | HR (95% CI) | 0.51 (0.35, 0.75) | | | |
| Age at | Number of events^{b}/total | 121/269 | (45.0) | 167/300 | (55.7) |
| randomisation: | number of patients (%) | | | | |
| ≥65 years | Median rPFS (months) (95% CI) | 21.95 | (19.32, 25.17) | 16.66 | (13.93, 19.35) |

| | |
|---|---|
| HR (95% CI) | 0.78 (0.62, 0.98) |

HRRm status based on ctDNA test (FoundationOne Liquid CDx)

| HRRm^{c} | Number of events^{b}/total | 42/98 | (42.9) | 66/100 | (66.0) |
|---|---|---|---|---|---|
| | number of patients (%) | | | | |
| | Median rPFS (months) (95% CI) | NC | (NC, NC) | 13.63 | (9.30, 16.59) |
| | HR (95% CI) | 0.54 (0.36, 0.79) | | | |
| Non-HRRm^{d} | Number of events^{b}/total | 117/269 | (43.5) | 147/267 | (55.1) |
| | number of patients (%) | | | | |
| | Median rPFS (months) (95% CI) | 24.11 | (19.35, 27.63) | 18.96 | (14.16, 21.19) |
| | HR (95% CI) | 0.76 (0.59, 0.97) | | | |
| HRRm Unknown^{e} | Number of events^{b}/total | 9/32 | (28.1) | 13/30 | (43.3) |
| | number of patients (%) | | | | |
| | Median rPFS (months) (95% CI) | NC | (NC, NC) | NC | (NC, NC) |

| | |
|---|---|
| HR (95% CI) | 0.62 (0.26, 1.44) |

HRRm status based on tumour tissue test (FoundationOne CDx)

| HRRm^{c} | Number of events^{b}/total | 22/62 | (35.5) | 37/56 | (66.1) |
|---|---|---|---|---|---|
| | number of patients (%) | | | | |
| | Median rPFS (months) (95% CI) | NC | (NC, NC) | 16.62 | (10.84, 19.38) |
| | HR (95% CI) | 0.44 (0.26, 0.74) | | | |
| Non-HRRm^{d} | Number of events^{b}/total | 94/207 | (45.4) | 113/210 | (53.8) |
| | number of patients (%) | | | | |
| | Median rPFS (months) (95% CI) | 22.54 | (17.58, 27.60) | 16.59 | (13.83, 21.19) |
| | HR (95% CI) | 0.81 (0.62, 1.07) | | | |
| HRRm Unknown^{e} | Number of events^{b}/total | 52/130 | (40.0) | 76/131 | (58.0) |
| | number of patients (%) | | | | |
| | Median rPFS (months) (95% CI) | 24.84 | (17.48, NC) | 16.39 | (13.77, 21.88) |

| | |
|---|---|
| HR (95% CI) | 0.64 (0.45, 0.90) |

^{a}HR and 95% CI from a Cox Proportional Hazards model as used for the primary analysis. HR < 1 favors olaparib + abiraterone acetate. CI calculated using the profile likelihood method;
^{b}Progression, as assessed by the investigator, is defined by RECIST 1.1 and/or PCWG-3 or death (by any cause in the absence of progression) regardless of whether the patient withdraws from randomised therapy or receives another anti-cancer therapy prior to progression. Each subgroup analysis was performed using a Cox Proportional Hazards model that contains a term for treatment, factor and treatment by factor interaction.
^{c}Defined as any deleterious or suspected deleterious HRR gene mutation detected.
^{d}Defined as no deleterious or suspected deleterious HRR gene mutation detected.
^{e}Test failed/sample not analysed.
bd, twice daily; CDx, companion diagnostic; CI, confidence interval; ctDNA, circulating tumour DNA; DCO, data cut-off; FAS, full analysis set; HR, hazard ratio; HRRm, homologous recombination repair gene mutation; NC, not calculated; qd, once daily; rPFS, radiological progression-free survival.

A phase II trial of the PARP inhibitor veliparib in combination with abiraterone acetate vs. abiraterone acetate found no significant difference in efficacy outcomes for patients with mCRPC when veliparib was added to abiraterone acetate treatment (Hussain M et al. J Clin Oncol 2018; 36:991-9). Data from the present study show an imaging-based progression-free survival benefit in patients unselected by HRRm and support a treatment benefit in the HRRm and non-HRRm subgroups.

A retrospective analysis demonstrated a strong correlation between PFS2 and overall survival, supporting the use of PFS2 to measure long-term clinical benefit when overall survival cannot be assessed. Although our overall survival data were 28.6% mature, there was a trend towards improved overall survival with olaparib and abiraterone acetate and the PFS2 results were supportive of longer-term improvement in efficacy outcomes. The Kaplan-Meier curves for both endpoints show a similar pattern, with separation occurring earlier for time to second progression-free survival or death.

There were more adverse events in the abiraterone acetate and olaparib arm of the trial, particularly anemia. However, the adverse-event profile for the abiraterone acetate and olaparib arm was consistent with their known individual toxicity profiles and does not suggest that combination therapy increases the toxicity of either drug. In the phase II trial of abiraterone acetate and olaparib vs. abiraterone acetate and placebo in mCRPC, more patients had cardiovascular events in the abiraterone acetate and olaparib arm. Reassuringly, in the present study, a difference in cardiovascular events was not observed, suggesting that the previously observed imbalance may have been due to the small population size of the phase II trial. There was a numerical imbalance in pulmonary events between the trial arms, and one event was fatal in the abiraterone acetate and olaparib arm, but no events led to discontinuation of olaparib or abiraterone acetate treatment. Pulmonary events have been observed in trials of other PARP inhibitors in mCRPC (in the TALAPRO-1 trial 6% of patients had a pulmonary embolism). No similar imbalance in pulmonary events has been observed in olaparib trials in other tumor types. At present, the mechanism of this effect in the mCRPC population is unknown.

At interim analysis, abiraterone acetate and olaparib led to significantly longer imaging-based progression-free survival than abiraterone acetate and placebo in patients with mCRPC, enrolled irrespective of HRRm status, who had not received treatment in the first-line setting. TFST, PFS2, the positive trend for overall survival in this interim data cut and the exploratory endpoints of ORR and PSA response further support the treatment benefit of abiraterone acetate and olaparib over abiraterone acetate and placebo in the overall intention-to-treat patient population. Although there were more adverse events in the abiraterone acetate and olaparib arm, they were as expected for the individual drugs, were generally manageable by dose interruption and dose reduction, and no detriment to HRQoL was reported. These results demonstrate the clinical benefits of olaparib in combination with abiraterone acetate in a broad, HRRm unselected population of patients with mCRPC. Such a combined therapeutic effect could potentially change clinical practice in the first-line mCRPC setting.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

What is claimed is:

1. A method of treating metastatic castration-resistant prostate cancer (mCRPC) in a subject, wherein the subject is treatment naïve, the method comprising:
administering to the subject a therapeutically effective amount of 4-[(3-{[4-(cyclopropane-carbonyl)piperazine-1-yl]carbonyl}-4-fluorophenyl)methyl]-2H-phthalazin-1-one (olaparib), or a salt, hydrate, solvate, or prodrug thereof;
administering to the subject a therapeutically effective amount of (3β)-17-(3-pyridinyl) androsta-5,16-dien-3-yl acetate (abiraterone acetate), or a salt thereof,
wherein progression free survival is at least about 6 months greater than for subjects receiving abiraterone acetate alone.

2. The method according to claim 1, wherein the subject has not previously received taxane-based chemotherapy previously.

3. The method according to claim 2, wherein the taxane-based chemotherapy is docetaxel.

4. The method according to any one of claims 1 to 3, wherein the subject has not previously received new hormonal agent chemotherapy previously, optionally wherein the new hormonal agent is enzalutamide or abiraterone acetate or a salt thereof.

5. The method according to any one of claims 1 to 4, wherein the cancer metastasized to bone and/or lymph nodes.

6. The method according to any one of claims 1 to 5, wherein the metastasis is visceral.

7. The method according to any one of claims 1 to 6, wherein the cancer cells are wild-type at one or more homologous recombination repair (HRR) genes.

8. The method according to any one of claims 1 to 6, wherein the cancer cells comprise one or more HRR gene mutations.

9. The method according to claim 8, wherein the HRR gene mutation is selected from BRCA1, BRCA2, ATM, BRIP1, BARD1, CDK12, CHEK1, CHEK2, FANCL, PALB2, PPP2R2A, RAD51B, RAD51C, RAD51D, and RAD54L gene mutation; or wherein the cancer cells comprise a BRCA1, a BRCA2, and/or an ATM gene mutation; or wherein the cancer cells comprise a BRCA1 and/or a BRCA2 gene mutation.

10. The method according to any one of claims 1 to 6, wherein the cancer is wild-type at one or more HRR gene mutations, and wherein the subject previously did not receive docetaxel.

11. The method according to any one of claims 1 to 10, wherein the therapeutically effective amount of olaparib is in the range of about 400 to 800 mg per day.

12. The method according to any one of claims 1 to 10, wherein the therapeutically effective amount of olaparib is about 600 mg daily.

13. The method according to any one of claims 1 to 10, wherein the therapeutically effective amount of olaparib is about 300 mg twice daily.

14. The method according to any one of claims 1 to 13, wherein the therapeutically effective amount of abiraterone acetate or salt thereof is in the range of about 500 to 1500 mg daily.

15. The method according to any one of claims 1 to 13, wherein the therapeutically effective amount of abiraterone acetate or salt thereof is in the range of about 800 to 1200 mg daily.

16. The method according to any one of claims 1 to 13, wherein the therapeutically effective amount of abiraterone acetate or salt thereof is about 1000 mg daily, optionally administered orally once daily.

17. The method according to any one of claims 1 to 16, further comprising administering prednisone or prednisolone in an amount of about 10 mg daily, optionally in an amount of about 5 mg twice daily.

18. The method according to any one of claims 1 to 17, wherein progression free survival is about 6 to 18 months greater, or about 6 to 14 months greater, or about 6 to 12 months greater

19. The method according to any one of claims 1 to 17, wherein progression free survival is about 8 to 18 months greater, or about 8 to 14 months greater, or about 8 to 12 months greater.

20. The method according to any one of claims 1 to 19, further comprising, prior to administration of olaparib and abiraterone acetate or salt thereof, selecting the subject based on prior treatment.

21. The method according to claim 20, wherein selecting does not comprise the step of selecting on a status of HRR gene mutations in the cancer.

22. Olaparib, or a salt, hydrate, solvate, or prodrug thereof, for use in the treatment of prostate cancer in a subject, wherein said treatment comprises separate, sequential or simultaneous administration of said olaparib, or a hydrate, solvate, or prodrug thereof, and abiraterone acetate or a or salt thereof, to said subject, wherein the subject has not been selected for HRR gene mutations in the cancer.

23. Olaparib for use according to claim 22, wherein the subject has a progression free survival that is at least about 6 months greater than for subjects receiving abiraterone acetate or salt thereof alone.

24. Olaparib for use according to claim 22 or claim 23, wherein the cancer has metastasized.

25. Olaparib for use according to of claim 24, wherein the metastasis is to bone and/or lymph nodes.

26. Olaparib for use according to of claim 24, wherein the metastasis is visceral.

27. Olaparib for use according to of any one of claims 22 to 26, wherein the cancer is mCRPC.

28. Olaparib for use according to of any one of claims 22 to 27, wherein the cancer cells are wild-type at one or more HRR genes.

29. Olaparib for use according to of any one of claims 22 to 26, wherein the cancer cells comprise one or more HRR gene mutations.

30. Olaparib for use according to of any one of claims 22 to 26, wherein the cancer is mCRPC comprising one or more HRR gene mutations.

31. Olaparib for use according to of claim 29 or 30, wherein the HRR gene mutation is selected from BRCA1, BRCA2, ATM, BRIP1, BARD1, CDK12, CHEK1, CHEK2, FANCL, PALB2, PPP2R2A, RAD51B, RAD51C, RAD51D, and RAD54L gene mutation; or wherein the cancer cells comprise a BRCA1, a BRCA2, and/or an ATM gene mutation; or wherein the cancer cells comprise a BRCA1 and/or a BRCA2 gene mutation.

32. Olaparib for use according to any one of claims 22 to 31, wherein the prostate cancer is mCRPC and the subject is treatment naïve.

33. Olaparib for use according to claim 32, wherein the subject is did not receive taxane-based chemotherapy previously, optionally wherein the taxane-based chemotherapy is docetaxel.

34. Olaparib for use according to any one of claims 22 to 33, wherein the subject did not receive new hormonal agent chemotherapy previously, optionally wherein the new hormonal agent is abiraterone acetate or salt thereof.

35. Olaparib for use according to any one of claims 22 to 31, wherein the subject previously received taxane-based chemotherapy.

36. Olaparib for use according to any one of claims 22 to 31, wherein the subject previously received new hormonal agent chemotherapy, optionally wherein the new hormonal agent is enzalutamide or abiraterone acetate or salt thereof.

37. Olaparib for use according to any one of claims 22 to 28, wherein the cancer is mCRPC, which is wild-type at one or more HRR gene mutations, and wherein the subject previously did not receive docetaxel.

38. Olaparib for use according to any one of claims 22 to 37, administered in an amount in the range of about 400 to 800 mg per day.

39. Olaparib for use according to any one of claims 22 to 37, administered in an amount of about 600 mg daily.

40. Olaparib for use according to any one of claims 22 to 37, administered in an amount of about 300 mg twice daily.

41. Olaparib for use according to any one of claims 22 to 40, wherein abiraterone is administered in an amount in the range of about 500 to 1500 mg daily.

42. Olaparib for use according to any one of claims 22 to 40, wherein abiraterone is administered in an amount in the range of about 800 to 1200 mg daily.

43. Olaparib for use according to any one of claims 22 to 40, wherein abiraterone is administered in an amount of about 1000 mg daily, optionally administered orally once daily.

44. Olaparib for use according to any one of claims 22 to 43, further comprising administering prednisone or prednisolone in an amount of about 10 mg daily, optionally in an amount of about 5 mg twice daily.

45. Olaparib for use according to any one of claims 23 to 44, wherein progression free survival is about 6 to 18 months greater, or about 6 to 14 months greater, or about 6 to 12 months greater

46. Olaparib for use according to any one of claims 23 to 44, wherein progression free survival is about 8 to 18 months greater, or about 8 to 14 months greater, or about 8 to 12 months greater.
